(19) United States
(12) Patent Application Publication
(10) Pub. No.: US 2020/0353222 A1
(43) Pub. Date: Nov. 12, 2020

(54) Conductance Based Digital Blood Flash Indicator Device

(71) Applicant: Bard Access Systems, Inc., Salt Lake City, UT (US)

(72) Inventors: Arthur E. Jaquez, Salt Lake City, UT (US); William Eccles, North Salt Lake, UT (US)

(21) Appl. No.: 16/868,483

(22) Filed: May 6, 2020

Related U.S. Application Data
(60) Provisional application No. 62/845,159, filed on May 8, 2019.

(51) Int. Cl. A61M 25/06; A61M 5/32; A61M 25/00; A61M 5/34

(52) U.S. Cl. CPC A61M 25/0693; A61M 5/3293; A61M 25/0014; A61M 2205/6054; A61M 2205/581; A61M 2205/583; A61M 2205/582; A61M 5/34

(57) ABSTRACT

Embodiments disclosed herein are generally directed to blood flash detection systems and methods thereof. Embodiments include an indicator device including a needle and needle hub, defining a lumen and in fluid communication with a medical line. The needle hub includes a marker, such as an RFID tag, with electrodes extending to the lumen. When a fluid flow, for example blood, enters the lumen, an electrical connection between the electrodes is bridged, completing the circuit and activating the marker. The marker can then be responsive to an interrogation signal, and can provide a response signal. The response signal can be detected and interpreted by a detection device that indicates to a user that the vasculature has been accessed without the user directly observing the insertion site or the device.

### PRIORITY

This application claims the benefit of priority to U.S. Provisional Application No. 62/845,159, filed May 8, 2019, which is incorporated by reference in its entirety herein.

### SUMMARY

Briefly summarized, embodiments disclosed herein are directed to a conductance based digital blood flash indicator devices and methods thereof. The digital blood flash indicator device (“indicator device” or “device”) includes a marker, such as an RFID tag, disposed in a needle hub with leads, or electrodes, extending into a lumen of the cannula. When a fluid, e.g. blood, contacts the electrodes, the circuit of the marker is closed, which allows the RFID antenna to broadcast a response signal. In an embodiment, the marker can be a passive RFID tag and a detection device, such as an ultrasound probe, can constantly broadcast an interrogation signal and monitor for any response signal. The presence of the response signal can indicate the presence of the fluid within the lumen. Optionally, additional information can be encoded within the response signal and can be interpreted by the detection device and displayed to the user.

Disclosed herein is an indicator device including, a needle supported by a needle hub, and defining a device lumen extending from a distal end of the needle to a proximal end of the needle hub, and a first marker including a first pair of electrodes extending through a wall of the needle hub to the device lumen, wherein a first fluid disposed within the device lumen contacts the first pair of electrodes and bridges a gap therebetween transitioning the first marker from an inactive state to an activated state.

In some embodiments, the marker is a passive RFID chip configured to receive an interrogation signal that induces the passive RFID chip in the activated state to provide a response signal. The blood flash indicator device is communicatively coupled with a detection device, the detection device configured to provide the interrogation signal. The detection device is configured to receive and interpret the response signal and provide an alert to a user. The alert includes one of an audio, visual, and tactile alert. The alert includes one of information about the indicator device, information about the first fluid, or instructions for the user. The marker is an active RFID chip and is configured to provide a response signal in the activated state. The marker is configured to provide a first response signal when the first fluid contacts the first pair of electrodes, and provides a second response signal, different from the first response signal, when a second fluid contacts the first pair of electrodes, the second fluid being different from the first fluid.

In some embodiments, the indicator device further includes a second marker including a second pair of electrodes extending to the device lumen and configured to transition to an activated state when the first fluid contacts the second pair of electrodes, wherein the second pair of electrodes disposed within the device lumen are in a longitudinally spaced apart relationship from the first pair of electrodes. In some embodiments, the indicator device further includes a second marker including a second pair of electrodes extending to the device lumen and configured to transition to an activated state when a second fluid contacts the second pair of electrodes, the second fluid being different from the first fluid. The needle hub includes a connector disposed at the proximal end, the connector providing fluid communication between the device lumen and at least one of a medical line, an I.V. fluid line, and a syringe.

Also disclosed is a system for confirming vascular access, the system including, an indicator device defining a lumen and including a needle, a needle hub, and a marker including a pair of electrodes extending to the lumen, and a detection device configured to provide an interrogation signal, wherein a fluid disposed within the lumen contacts the pair of electrodes transitioning the marker to an activated state, the interrogation signal inducing the marker in the activated state to provide a response signal.

In some embodiments, the marker is a passive RFID chip and the response signal is a reflected interrogation signal. The response signal includes additional information. The additional information includes at least one of information about the indicator device or information about the fluid. The detection device receives and interprets the response signal and provides an alert to a user, the alert including one of an audio, visual, and tactile alert.

Also disclosed is a method of accessing a vasculature of a patient including, obtaining an indicator device having, a needle supported by a needle hub, and a marker disposed on an outer surface of the needle hub, the marker including a pair of electrodes extending through a side wall of the needle hub to an interior of the needle hub, advancing a distal tip of the needle into the patient, and confirming that the distal tip of the needle is in the vasculature of the patient, wherein fluid from the vasculature contacts the pair of electrodes to activate the marker and induce a response signal from the marker.

In some embodiments, the marker is a passive RFID chip, and wherein the response signal is further induced by receiving an interrogation signal from a detection device. The detection device is an ultrasound imaging device, and wherein the detection device receives the response signal and provides an alert to a user indicating that the vasculature has been accessed. The marker is an active RFID chip and the response signal includes additional information about one of the indicator device or the fluid.

### DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1A shows a perspective view of a conductance based indicator device, in accordance with embodiments disclosed herein.

FIG. 1B shows a plan view of the device of FIG. 1A, in accordance with embodiments disclosed herein.

FIG. 1C shows a side view of the device of FIG. 1A, in accordance with embodiments disclosed herein.

FIG. 1D shows a proximal end perspective view of the device of FIG. 1A, in accordance with embodiments disclosed herein.

FIG. 2 shows an exploded view of a conductance based indicator device, in accordance with embodiments disclosed herein.

FIGS. 3A-3B show cross-sectional side views of conductance based indicator devices, in accordance with embodiments disclosed herein.

FIGS. 4A and 4B show an exemplary environment of use for a conductance based indicator device, in accordance with embodiments disclosed herein.

### DETAILED DESCRIPTION

Reference will now be made to figures wherein like structures will be provided with like reference designations. It is understood that the drawings are diagrammatic and schematic representations of exemplary embodiments of the present invention, and are neither limiting nor necessarily drawn to scale.

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” “upper,” “lower,” “underside,” “upperside” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. Also, the words “including,” “has,” and “having,” as used herein, including the claims, shall have the same meaning as the word “comprising.”

With respect to “proximal,” a “proximal portion” or a “proximal end portion” of, for example, a needle disclosed herein includes a portion of the needle intended to be near a clinician when the needle is used on a patient. Likewise, a “proximal length” of, for example, the needle includes a length of the needle intended to be near the clinician when the needle is used on the patient. A “proximal end” of, for example, the needle includes an end of the needle intended to be near the clinician when the needle is used on the patient. The proximal portion, the proximal end portion, or the proximal length of the needle can include the proximal end of the needle; however, the proximal portion, the proximal end portion, or the proximal length of the needle need not include the proximal end of the needle. That is, unless context suggests otherwise, the proximal portion, the proximal end portion, or the proximal length of the needle is not a terminal portion or terminal length of the needle.

With respect to “distal,” a “distal portion” or a “distal end portion” of, for example, a needle disclosed herein includes a portion of the needle intended to be near or in a patient when the needle is used on the patient. Likewise, a “distal length” of, for example, the needle includes a length of the needle intended to be near or in the patient when the needle is used on the patient. A “distal end” of, for example, the needle includes an end of the needle intended to be near or in the patient when the needle is used on the patient. The distal portion, the distal end portion, or the distal length of the needle can include the distal end of the needle; however, the distal portion, the distal end portion, or the distal length of the needle need not include the distal end of the needle. That is, unless context suggests otherwise, the distal portion, the distal end portion, or the distal length of the needle is not a terminal portion or terminal length of the needle.

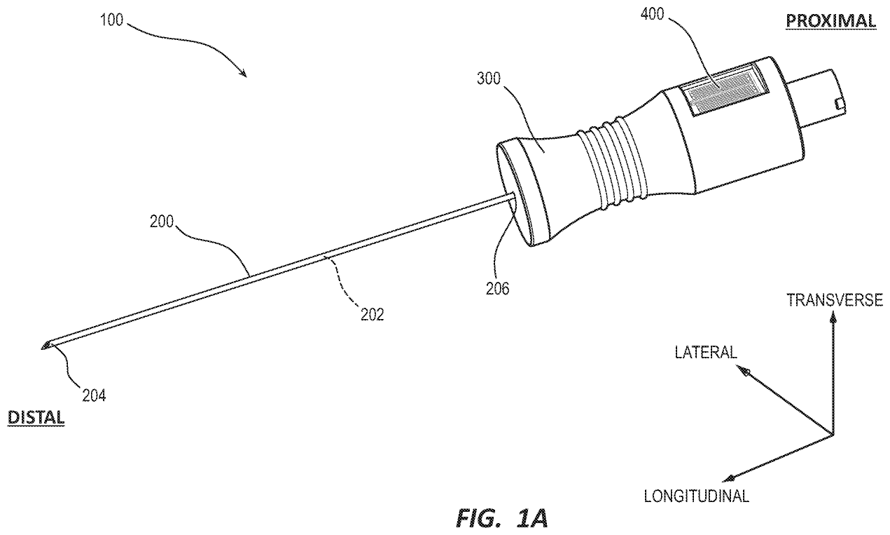

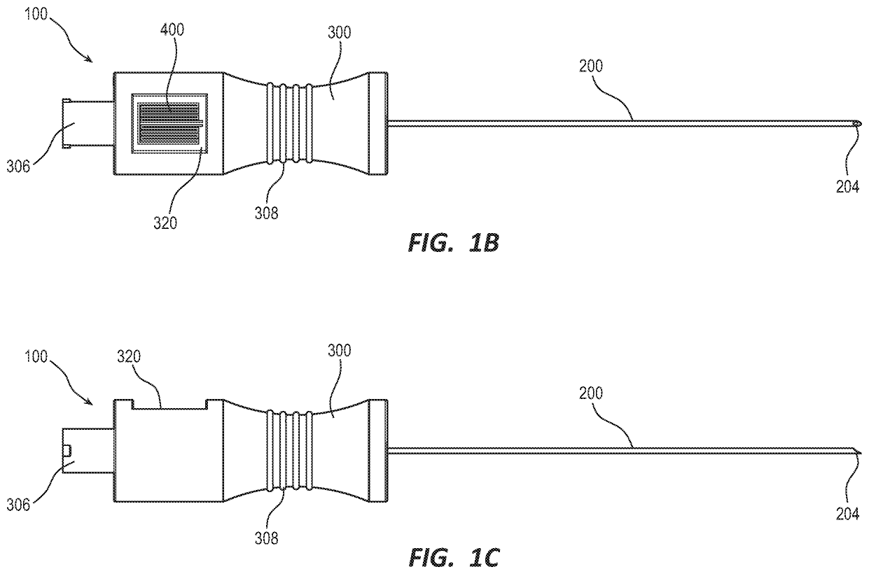

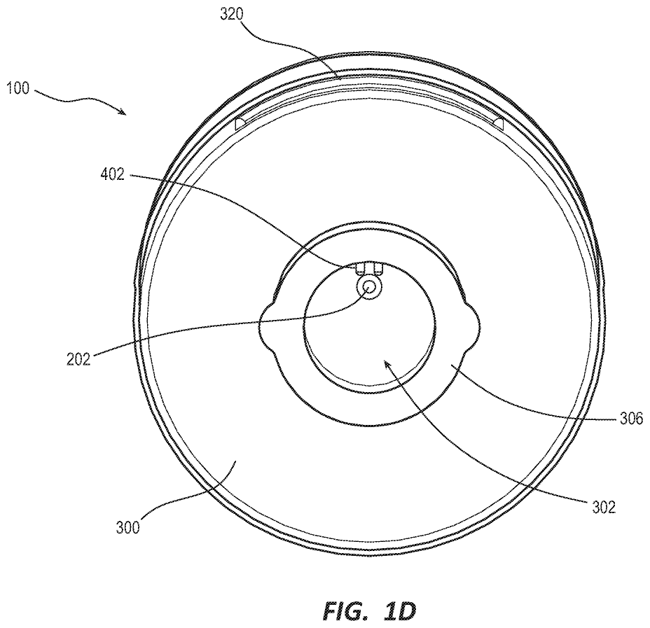

To assist in the description of embodiments disclosed herein, the following coordinate terms are used (see FIG. 1A). A “longitudinal axis” is generally parallel to the axis of a needle of the device. A “lateral axis” is normal to the longitudinal axis. A “transverse axis” extends normal to both the longitudinal and lateral axes. In addition, as used herein, “the longitudinal direction” refers to a direction substantially parallel to the longitudinal axis; “the lateral direction” refers to a direction substantially parallel to the lateral axis; and “the transverse direction” refers to a direction substantially parallel to the transverse axis. The term “axial” as used herein refers to the axis of the needle, and therefore is substantially synonymous with the term “longitudinal” as used herein.

Unless defined otherwise, all technical and scientific terms used herein have the same meaning as commonly understood by those of ordinary skill in the art.

Briefly summarized, embodiments herein are generally directed to a blood flash detection systems and methods thereof. Embodiments include an indicator device, such as a needle and needle hub, defining a lumen and in fluid communication with a medical line. The needle hub includes a marker, such as an RFID tag, with electrodes disposed within the lumen. When a fluid flow, e.g. blood, enters the lumen, the electrical connection between the electrodes is bridged, completing the circuit and activating the marker. The marker is then responsive to an interrogation signal, and can provide a response signal. The response signal is detected and interpreted by a detection device that indicates to a user that the vasculature has been accessed without the user directly observing the insertion site or the device. Embodiments herein further describe additional aspects of the conductance based digital blood flash device and methods of use thereof.

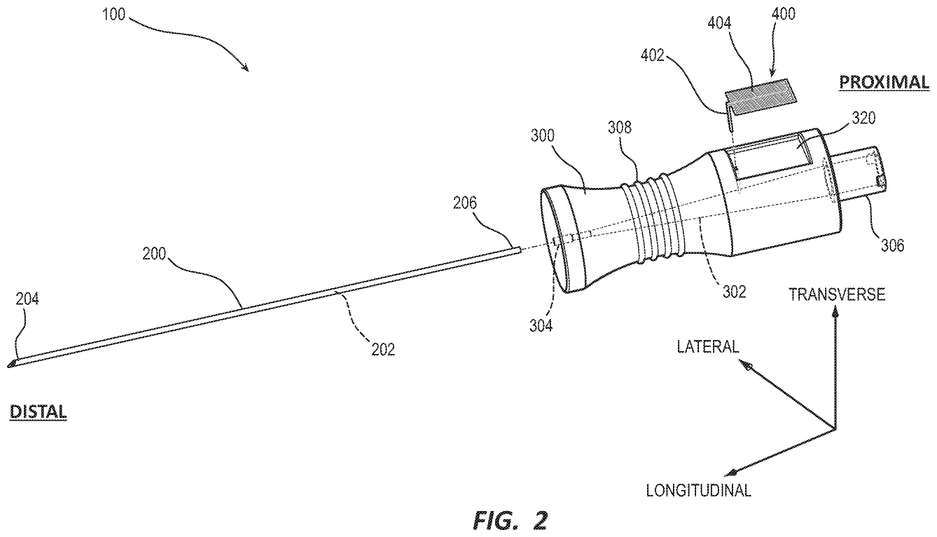

FIGS. 1A-2 depict various details of a conductance based blood flash indicator device (“indicator device” or “device”) 100 in accordance with embodiments of the present disclosure. The device 100 can generally include a cannula, such as needle 200, a needle hub 300, and a marker 400. In an embodiment, the marker 400 can include a passive RFID tag or an active RFID tag, although it will be appreciated that the marker 400 can utilize various different communication modalities, other than radio frequency (RF), including electromagnetic (EM), microwave, infrared (IR), optical, x-ray, magnetic, acoustic (e.g. ultrasound) or similar suitable modalities.

The needle 200 can define a lumen 202 extending from a needle tip 204, disposed at a distal end, to a proximal end 206 of the needle 200. The needle 200 is supported by a needle hub 300, where a proximal end 206 of the needle is received within a needle housing 304, which fluidly connects a needle lumen 202 with a hub lumen 302. The needle hub lumen 302 extends from the needle housing 304 at a distal end of the needle hub 300, to a connector 306 at a proximal end. The hub lumen 302 can define a substantially cylindrical shape. In an embodiment, the hub lumen 302 defines a tapered shape extending from a first lumen diameter to a second lumen diameter that is less than the first lumen diameter. As such, the hub lumen 302 can provide a transition between a lumen diameter of the connector 306 (i.e. a first lumen diameter), and a lumen diameter of the needle lumen 202 (i.e. a second lumen diameter). The connector 306 can be coupled to various additional devices such as medical lines, intravenous (I.V.) fluid lines, syringes, or the like, providing fluid communication therebetween. Exemplary embodiments of connector 306 include male or female luer locks, spin locks, twist locks, bayonet connectors, or the like. The needle hub 300 can further include gripping features 308 to facilitate manipulation of the device 100. As used herein, the needle lumen 202 and the hub lumen 302 can be collectively termed a “device lumen”.

In an embodiment, the blood flash device 100 can further include a marker 400. The marker 400 can be disposed on an outer surface of the needle hub 300. In an embodiment, the marker 400 can be disposed within a recess 320. The marker 400 can be secured to the hub 300 using adhesives, bonding, welding, or similar suitable attachment means. In an embodiment, the marker 400 is disposed within recess 320 and overlaid with an epoxy resin or similar suitable material.

As shown in FIGS. 1D-2, in an embodiment, the marker 400 includes a pair of electrodes 402 extending from the marker 400 through a side wall of the needle hub 300, to a lumen of the device, e.g. needle hub lumen 302. The electrodes 402 can form part of a discontinuous electrical circuit 404. The circuit 404 can be completed when a gap between the electrodes is conductively bridged. Worded differently, conductively bridging the gap between the electrodes 402 can transition the discontinuous electrical circuit 404 to a continuous electrical circuit 404. For example, when a conductive fluid, e.g. blood, plasma, saline, interstitial fluid, or the like, enters the needle hub lumen 302, an electrical connection between the electrodes is bridged and the circuit 404 is completed, activating the marker 400.

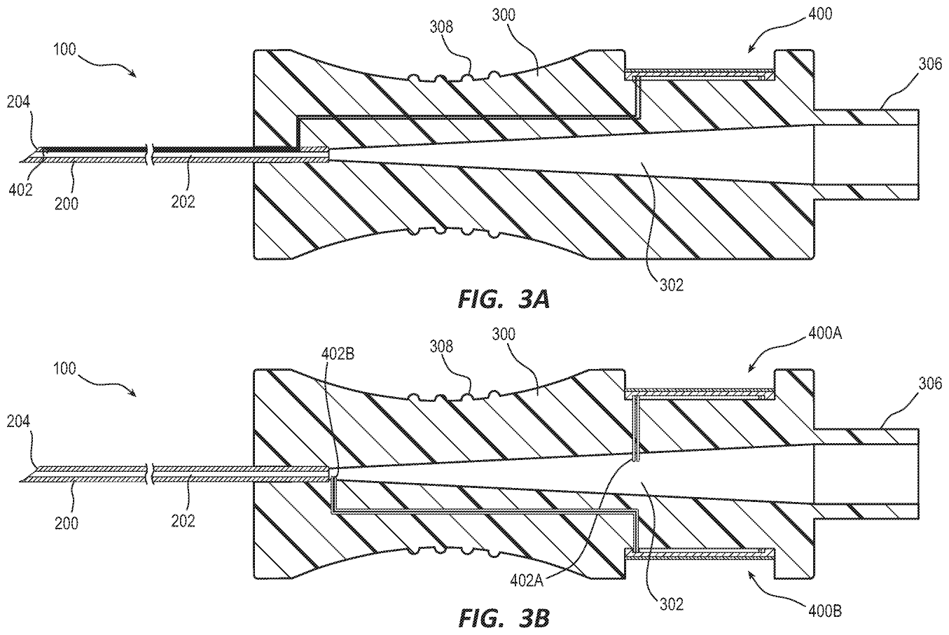

As shown in FIGS. 3A-3B, in an embodiment, the electrodes 402 can extend distally of the marker 400 through a wall portion of the needle hub 300, needle 200, or combinations thereof, before entering the device lumen. For example, as shown in FIG. 3A, the electrodes 402 can enter the device lumen at a distal portion of the needle lumen 202. As shown in FIG. 3B, the electrodes 402 can enter the device lumen at a distal portion of the hub lumen 302 (e.g. electrodes 402B) or a proximal portion of the hub lumen 302 (e.g. electrodes 402A). These and other combinations of longitudinal positions of the electrodes 402 are contemplated to fall within the scope of the present invention. As such, the marker 400 can be activated and indicate to a user when a fluid flow has reached a particular portion of the device 100. Advantageously, electrodes positioned closer to a needle tip 204 can provide faster response times for detecting the presence of a fluid flow. Advantageously, electrodes positioned closer to the marker 400 provide reduced manufacturing complexities and associated costs.

As shown in FIG. 3B, in an embodiment, the device 100 can include two or more markers 400, each including a pair of electrodes 402. For example, the device 100 can include a first marker 400A that includes a first pair of electrodes 402A and a second marker 400B that includes a second pair of electrodes 402B. In an embodiment, the first pair of electrodes 402A and the second pair of electrodes 402B can extend to a similar longitudinal position within the device lumen. In an embodiment, the first pair of electrodes 402A and the second pair of electrodes 402B can extend to different longitudinal positions within the device lumen. As such, the two or more markers 400A, 400B, can indicate a relative position of a fluid flow through the device lumen.

In an embodiment, the first marker 400A and the second marker 400B can be configured to detect the same fluid or different types of fluids. For example, a marker 400 can be configured to respond differently accordingly the different conductance of the fluid completing the circuit. Alternatively, a first marker 400A can be configured to respond to the presence of a first fluid, and a second marker 400B can be configured to respond to the presence of a second fluid, different from the first fluid. A first fluid or the second fluid can include oxygenated blood, deoxygenated blood, plasma, interstitial fluid, saline, combinations thereof, or the like. For example, a first marker 400A can be configured to respond to the presence of oxygenated blood, and can indicate if device has accessed an artery, and a second marker 400B can be configured to respond to deoxygenated blood and can indicate if the device 100 has accessed a vein. Advantageously, the marker(s) 400 can indicate if the device has accessed a target vessel correctly (e.g. a vein), or has incorrectly accessed a non-target vessel (e.g. artery), a surrounding tissue, or the like. Advantageously, the marker(s) 400 can be configured to detect the presence and subsequent absence of a target fluid, e.g. deoxygenated blood, which might indicate the device has initially accessed a target vessel, e.g. a vein and then been advanced too far and breached a far wall of the vein entering the surrounding tissue on the far side of the vessel, termed “backwalling” Such information, and the like, can be detected by the device 100 can communicated to the user, as discussed in more detail herein.

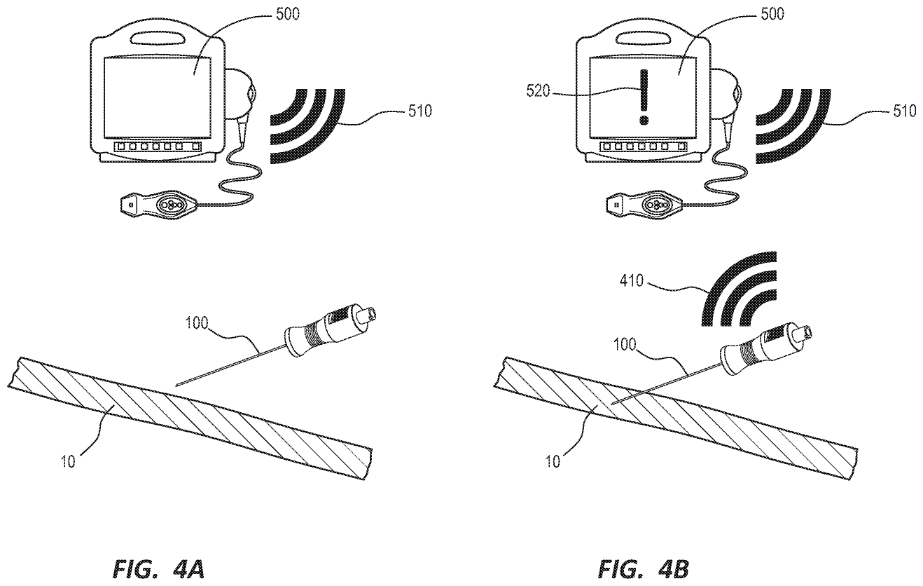

In an exemplary method of use, and as shown in FIGS. 4A-4B, the blood flash device 100 can be used to access a vasculature 10 of a patient. The blood flash device 100 can be communicatively coupled with a detection device 500. In an embodiment, the detection device 500 can be a standalone handheld device. In an embodiment, the detection device 500 can be incorporated as part of a larger system such as a hospital or clinic network, electronic medical record (EMR) network, robotic surgery systems, imaging systems, combinations thereof, or the like. In an embodiment the detection device can be included as part of an ultrasound imaging system, although other imaging systems are also contemplated such as x-ray fluoroscopy, PET, CAT, MRI, or the like.

The detection device 500 can provide an interrogation signal 510 that impinges on the blood flash device 100 and marker 400. In an embodiment the marker 400 can be a passive RFID tag. As shown in FIG. 4A in the absence of any fluid flow within the hub 300, the interrogation signal 510 can impinge on the marker 400 but fail to induce a response signal 410 from the marker 400. Worded differently, when a distal tip of the needle 204 has not entered a vasculature 10, no response signal can be provided 410, despite receiving a consistent interrogation signal 510.

A clinician can then manipulate the needle hub 300 to advance the needle tip 204 into the vasculature 10 of the patient, optionally assisted by ultrasound imaging guidance. When the needle tip 204 enters the vasculature of the patient, blood can flow proximally through the lumen of the device 100. The blood flow in the device lumen can contact the pair of electrodes 402 creating an electrical connection therebetween. As such, the electrical connection between the electrodes 402 can transition the circuit 404 from a discontinuous circuit to a continuous circuit, and can activate the marker 400.

As shown in FIG. 4B, with the marker 400 activated, the interrogation signal 510 impinging on the marker 400 can then induce a response signal 410. The response signal 410 can be received and interpreted by the detection device 500 and indicate to a user that a blood flash has been detected and the needle tip 204 has accessed the vasculature 10. In an embodiment, the response signal 410 is a reflected signal that is substantially the same as the interrogation signal 510. As such a presence or absence of the response signal 410 provides the information that a fluid flow is present within the device lumen.

In an embodiment, the response signal 410 can be a different signal from that of the interrogation signal 510. In an embodiment, the marker 400 can include additional information and encode this information in the response signal 410. For example, the additional information can be stored on the marker 400 and include identifying information about the device 100, such as make, model, serial number, or details about components of the device 100 such as needle gauge, combinations thereof, and the like.

In an embodiment, the additional information can include information about the type of fluid disposed within the needle hub, such as blood, plasma, interstitial fluid, peritoneal fluid, Ringer's solution, medications, or the like. For example, as discussed herein, different types of fluid can provide different conductance profiles which in turn be determined by the marker 400 and can alter the response signal 510 accordingly. In an embodiment, the device 100 can include two or more markers, e.g. marker 400A and 400B. A first marker 400A can be configured to detect a first fluid type, or group of fluid types, and a second marker 400B can be configured to detect a second fluid type, or group of fluid types, different from the first fluid type, or group. As such, each of the markers 400A, 400B can activate and provide a response signal when a corresponding fluid type is present. These differing response signals 510 can be detected and interpreted by the detection device 500, and the information can be provided to the user.

In an embodiment, the detection device 500 can interpret the response signal 410 and provide an alert 520 to the user. The alert 520 can be a visual, auditory, or tactile alert, or combinations thereof. Optionally, the alert 520 can include additional information about the device 100, fluid detected, instructions for the user, combinations thereof, or the like. Advantageously, the detection device 500 can provide an alert 520 to the user by way of various modalities, without the user having to directly observe the insertion site, the device 100, or combinations thereof. For example, a clinician accessing a vasculature under imaging requires focusing on a screen that is separate from the insertion site. Typically, in order to determine vasculature access, the clinician must periodically divert their attention from the imaging monitor to the insertion site. In the present invention, the device 100 can indicate to the user that the vasculature has been accessed without the user diverting their attention. The alert 520 can be provided as an image, icon, instructions, or notification on the imaging screen. The alert 520 can be an audible tone, flashing light, display instruction, or the like, to indicate that a vasculature has been accessed. The alert 520 can be a tactile or haptic indication, in the form of a strike or vibration that a user can feel, when a vasculature has been accessed, e.g. provided by way of the needle hub 300, gripping feature 308, or the like.

Further, the alert 520 can be transmitted, by way of a network, so that other individuals can be notified of the vascular access, such as those assisting or observing the clinician operating the device. Similarly, the device 100 and detection device 500 can provide an alert 520 to indicate if the device 100 has accessed a vasculature incorrectly. For example, the alert 520 can indicated that an artery has been accessed, instead of a vein, by detecting the presence of oxygenated blood. The alert 520 can indicate the device has accessed an interstitial portion and not yet accessed the target vessel 10. The alert 520 can indicate if the vessel 10 had been accessed and then backwalled, passing through a far wall of the vessel 10 to a surrounding tissue. These and similar combinations of alerts 520 are contemplated to fall within the scope of the present invention.

Advantageously, the marker 400 can include a passive RFID tag that does not require additional power sources, circuitry and components to provide a response signal 410. This reduces the size and weight of the tag and the marker can be included on smaller medical devices. Further passive RFID tags can reduce manufacturing complexity and associated costs.

In an embodiment, the marker 400 can include an active RFID tag. As such, the marker 400 can further include a sensor, power source, circuitry components, combinations thereof and the like, to actively detect the presence of one or more types of fluid, e.g. blood, in the lumen of the device 100 and send a response signal 410 to the detection device 500. Advantageously, the active RFID tag can provide a larger range of the response signal 410 and does not require an interrogation signal in order to provide a response signal.

Embodiments of the invention may be embodied in other specific forms without departing from the spirit of the present disclosure. The described embodiments are to be considered in all respects only as illustrative, not restrictive. The scope of the embodiments is, therefore, indicated by the appended claims rather than by the foregoing description. All changes that come within the meaning and range of equivalency of the claims are to be embraced within their scope.

What is claimed is:

1. An indicator device, comprising:
a needle supported by a needle hub, and defining a device lumen extending from a distal end of the needle to a proximal end of the needle hub; and
a first marker including a first pair of electrodes extending through a wall of the needle hub to the device lumen, wherein a first fluid disposed within the device lumen contacts the first pair of electrodes and bridges a gap therebetween transitioning the first marker from an inactive state to an activated state.

2. The indicator device according to claim 1, wherein the marker is a passive RFID chip configured to receive an interrogation signal that induces the passive RFID chip in the activated state to provide a response signal.

3. The indicator device according to claim 2, wherein the blood flash indicator device is communicatively coupled with a detection device, the detection device configured to provide the interrogation signal.

4. The indicator device according to claim 3, wherein the detection device is configured to receive and interpret the response signal and provide an alert to a user.

5. The indicator device according to claim 4, wherein the alert includes one of an audio, visual, and tactile alert.

6. The indicator device according to claim 4, wherein the alert includes one of information about the indicator device, information about the first fluid, or instructions for the user.

7. The indicator device according to claim 1, wherein the marker is an active RFID chip and is configured to provide a response signal in the activated state.

8. The indicator device according to claim 1, wherein the marker is configured to provide a first response signal when the first fluid contacts the first pair of electrodes, and provides a second response signal, different from the first response signal, when a second fluid contacts the first pair of electrodes, the second fluid being different from the first fluid.

9. The indicator device according to claim 1, further including a second marker including a second pair of electrodes extending to the device lumen and configured to transition to an activated state when the first fluid contacts the second pair of electrodes, wherein the second pair of electrodes disposed within the device lumen are in a longitudinally spaced apart relationship from the first pair of electrodes.

10. The indicator device according to claim 1, further including a second marker including a second pair of electrodes extending to the device lumen and configured to transition to an activated state when a second fluid contacts the second pair of electrodes, the second fluid being different from the first fluid.

11. The indicator device according to claim 1, wherein the needle hub includes a connector disposed at the proximal end, the connector providing fluid communication between the device lumen and at least one of a medical line, an I.V. fluid line, and a syringe.

12. A system for confirming vascular access, the system comprising:
an indicator device defining a lumen and including a needle, a needle hub, and a marker including a pair of electrodes extending to the lumen; and
a detection device configured to provide an interrogation signal, wherein a fluid disposed within the lumen contacts the pair of electrodes transitioning the marker to an activated state, the interrogation signal inducing the marker in the activated state to provide a response signal.

13. The system of claim 12, wherein the marker is a passive RFID chip and the response signal is a reflected interrogation signal.

14. The system of claim 12, wherein the response signal includes additional information.

15. The system of claim 14, wherein the additional information includes at least one of information about the indicator device or information about the fluid.

16. The system of claim 12, wherein the detection device receives and interprets the response signal and provides an alert to a user, the alert including one of an audio, visual, and tactile alert.

17. A method of accessing a vasculature of a patient, comprising:
obtaining an indicator device, comprising:
a needle supported by a needle hub; and
a marker disposed on an outer surface of the needle hub, the marker including a pair of electrodes extending through a side wall of the needle hub to an interior of the needle hub;
advancing a distal tip of the needle into the patient; and
confirming that the distal tip of the needle is in the vasculature of the patient, wherein fluid from the vasculature contacts the pair of electrodes to activate the marker and induce a response signal from the marker.

18. The method of claim 17, wherein the marker is a passive RFID chip, and wherein the response signal is further induced by receiving an interrogation signal from a detection device.

19. The method of claim 18, wherein the detection device is an ultrasound imaging device, and wherein the detection device receives the response signal and provides an alert to a user indicating that the vasculature has been accessed.

20. The method of claim 17, wherein the marker is an active RFID chip and the response signal includes additional information about one of the indicator device or the fluid.
